(19) United States
(12) Patent Application Publication
(10) Pub. No.: US 2007/0065924 A1
(43) Pub. Date: Mar. 22, 2007

(54) Isolated polynucleotide sequence with IRES activity

(75) Inventors: Tzong-Yuan Wu, Panchiao City (TW); Chung-Hsiung Wang, Tainan City (TW); Chih-Yu Wu, Keelung City (TW); Ying-Ju Chen, Shinyuan Township (TW)

Correspondence Address: THOMAS, KAYDEN, HORSTEMEYER & RISLEY, LLP, 100 GALLERIA PARKWAY, NW, STE 1750, ATLANTA, GA, 30339-5948, US

(73) Assignees: Chung Yuan Christian University; National Taiwan University

(21) Appl. No.: 11/524,428

(22) Filed: Sep. 20, 2006

(30) Foreign Application Priority Data
Sep. 21, 2005 (TW) 94132691

(51) Int. Cl. C12Q 1/68; C12P 19/34

(52) U.S. Cl. 435/91.1; 435/6

(57) ABSTRACT

Provided herein is an isolated polynucleotide sequence with internal ribosome entry site (IRES) activity, which directs translation initiation in an insect expression system in a cap-independent manner. In particular, the invention relates to an isolated polynucleotide comprises the 5′ UTR of perina nuda Picorna-like virus (PnV) that possesses IRES activity. Methods of identifying a polynucleotide with IRES activity and methods of expressing at least two polypeptides in an insect system are also disclosed herein.

### RELATED APPLICATIONS

The present application is based on, and claims priority from, Taiwan Application Serial Number 94132691, filed Sep. 21, 2005, the disclosure of which is hereby incorporated by reference herein in its entirety.

### BACKGROUND

1. Field of Invention

The present invention relates to a polynucleotide that affects gene expression in translational level, in particular to a polynucleotide derived from an insect picorna-like RNA virus, which has an effect on the initiation of mRNA translation in an insect expression system.

2. Description of Related Art

Through biotechnology, hundreds of heterologous proteins can be expressed in cells of insects or vertebrates by using viral expression vectors for mass-production. However, a complex protein, such as a membrane or secretory protein, is often composed of several different subunits. For example, a secretory antibody is composed of light chains and heavy chains that connected via disulfide bonds and non-covalent bonds; and an acetylcholine receptor, which is a pentamer composed of α, β, γ and δ subunits. For a complex protein to be made, all subunits of the protein need to be expressed in the same cell by using several vectors respectively encoding each of the subunits before assembling into a functional protein. A bi-cistronic or multi-cistronic expression vector can be very useful for this purpose.

The discovery of the internal ribosome entry site (IRES) brings expression vectors to a new era of expression. Internal ribosome entry site (IRES) was originally discovered in poliovirus of the picornavirus family (Pelletier, J. and Sonenberg, Nature, 334: 320-325, 1988). Internal ribosome entry site in a RNA molecule forms a specific secondary or tertiary structure to direct translational initiation without prior scanning of the mRNA with 40S subunits, and is termed as “cap-independent translation” or “IRES-dependent translation”. Currently, IRES elements have been widely used in mammalian expression vectors for bi-cistronic or multi-cistronic expression, such as a bi-cistronic expression vector containing the IRES of the Encephalomyocarditis virus. However, an insect-based expression vector with IRES activity for bi-cistronic or multi-cistronic expression has not yet been established.

Over 25 small RNA-containing viruses have been found in various insects and were termed “insect picorna-like virus” on the basis of the similarity to mammalian picornavirus in structure, composition of capsid and the biophysical properties of their RNA genome (C. M. Fauquet et al., Virus Taxonomy: 8th Report of the International Committee on Taxonomy of Viruses; Academic Press, San Diego, Calif., 779-787, 2005). These insect picorna-like viruses are currently divided into two major groups according to their genome organization and the phylogenetic relationship derived from RNA-dependent RNA polymerase (RdRp). One group of insect picorna-like viruses that used to be termed “Cricket paralysis-like virus” has recently been categorized in a new family “Dicistroviridae” (C. M. Fauquet et aL, Virus Taxonomy: 8th Report of the International Committee on Taxonomy of Viruses; Academic Press, San Diego, Calif., 783-788, 2005). Members of the Dicistroviridae family are characterized in having two non-overlapping open reading frames (ORFs) spaced apart by an intergenic region that functions as an IRES. Furthermore, non-structural proteins are encoded by the 5′-proximal ORF, whereas capsid proteins are encoded by 3′-proximal ORF. The other group of insect picorna-like viruses containing a monocistronic genome represents a new genus “Iflavirus”, such as “perina nuda Picorna-like virus” (PnV) (Wu et al., Virology, 294: 312-323). The genus Iflavirus, much like mammalian small RNA viruses, is distinct from Dicistroviridae in that all members of the Iflavirus genus have a single large open reading frame (ORF) to encode both structural and non-structural proteins. Like picornaviruses of mammalia, the capsid proteins of an insect small RNA virus are encoded by 5′ regions of the genome, and the non-structural proteins are encoded by 3′ parts.

In the present invention, the inventors identified a polynucleotide sequence with IRES activity located at the 5′ untranslated region (5′ UTR) of perina nuda Picorna-like virus genome, and thereby completing this invention, and a bi-cistronic baculovirus expression vector for an insect-based expression system (e.g. an insect, an insect cell or insect tissue) may be constructed by using this newly identified 5′ UTR sequence in the PnV genome.

### SUMMARY

In one aspect, this invention provides an isolated polynucleotide sequence with IRES activity (also termed as “IRES sequence”), which directs an mRNA containing the isolated IRES sequence therein to undergo cap-independent translation initiation.

In another aspect, this invention provides a method of screening a polynucleotide sequence with IRES activity from the genome of a small RNA virus.

In an alternative aspect, the invention provides a method of simultaneously expressing at least two proteins or peptides in a single insect cell.

In an embodiment according to the present invention, an isolated polynucleotide sequence with IRES activity is provided. The polynucleotide sequence with IRES activity comprises nucleotide sequences of the 5′ UTR of perina nuda Picorna-like virus genome sufficient to direct Cap-independent translation.

The present invention provides a method of expressing at least two polypeptides or proteins in an insect-based expression system comprising steps of: constructing a viral expression vector to comprise a first cistron operatively linked to a promoter of the viral expression vector, a polynucleotide sequence with IRES activity operatively linked to the first cistron and a second cistron operatively linked to the polynucleotide sequence with IRES activity in sequence; infecting the insect-based expression system with the viral expression vector; and detecting the expression of the first and second cistrons in the insect-based expression system.

The present invention also provides a kit comprising at least one polynucleotide sequence with IRES activity derived from the 5′ UTR of the perina nuda Picorna-like virus (PnV) genome and an instruction manual for using the IRES sequence.

It is to be understood that both the foregoing general description and the following detailed description are examples and are intended to provide further explanation of the invention as claimed.

### BRIEF DESCRIPTION OF THE DRAWINGS

The file of this patent contains at least one drawing executed in color. Copies of this patent with color drawing(s) will be provided by the Patent and Trademark Office upon request and payment of the necessary fee.

The invention will be illustrated with respect to the accompanying figures and examples, which serve to illustrate the present invention but are not binding thereon, wherein:

FIG. 1 is a schematic construction of baculovirus expression vectors used in this study, comprising a reporter gene, an IRES sequence or the 5′ UTR of perina nuda Picorna-like virus and another reporter gene following the promoter (P_{PH}) in sequence;

FIG. 2 is a Northern blot illustrating that the total RNAs of the Sf21 cells on day 4 after the infection with or without the vAcD-Pnir-E recombinant virus;

FIGS. 3A-3B illustrating the expression of DsRed and EGFP genes in Sf21, SE, and SL-1A insect cells infected with vAcD-Pnir-E or vAcD-Crir-E;

FIG. 4 is a Western blot illustrating the protein expression levels of DsRed and EGFP genes in the vAcD-Pnir-E or vAcD-Crir-E infected SF21 insect cells;

FIG. 5 is the construction of baculovirus expression vectors used in this study, illustrating that a DNA fragment containing both PnV-5′ UTR and the first 22 codons of PnV-ORF are cloned into pBacDirE plasmid;

FIG. 6 illustrating the expression of DsRed and EGFP genes in insect cells infected with vAcD-Pnir-E (PnV) or vAcD-Pn539ir-E (PnV 5′539).

FIG. 7 illustrating the red and green fluorescence intensity of DsRed and EGFP proteins in cells respectively infected with vAcD-Crir-E (CrPV), vAcD-Pnir-E (PnV), vAcD-Rhir-E (RhPV) or vAcD-Pn539ir-E (PnV 5′539);

FIGS. 8 is a Western blot illustrating the levels of DsRed and EGFP proteins in the SF21 insect cells respectively infected with recombinant viruses vAcD-Crir-E (CrPV), vAcD-Pnir-E (PnV), vAcD-Rhir-E (RhPV) or vAcD-Pn539ir-E (PnV 5′539);

FIGS. 9 shows the homology between two segments, 5′ end 1-393 nts of EoPV (SEQ. ID NO: 11) and 5′ end 84-476 nts of PnV (SEQ. ID NO: 1), with pairwise alignment by BioEdit 7.0 Software.

### DETAILED DESCRIPTION

In one aspect, the present invention provides a method of screening a virus genome, particularly, the perina nuda Picorna-like virus (PnV) genome, for a polynucleotide sequence with IRES activity. A candidate sequence with IRES activity can be selected according to various principles. For example, it is well accepted that when two RNAs, DNAs or proteins possess similar structures (e.g. similar in secondary or tertiary structure), it is highly likely that they may also possess similar functions and presumably similar sequences as well. Therefore, if a polynucleotide sequence is more than 70% identical to a known IRES sequence, such as 70%, 80%, 90%, 95% or more, the polynucleotide sequence may be considered as a candidate of an IRES element. For example, a candidate of an IRES sequence used in a study of IRES activity may be selected according to the sequence homology to a known IRES sequence by using well known softwares of sequence-alignment, e.g. BioEdit 7.0 or the nucleotide-nucleotide BLAST tool provided by GenBank website.

An IRES sequence candidate and reporter genes can be cloned into a baculovirus transfer vector by using any of the well-known plasmid construction techniques. Typically, these desired sequences and reporter genes are inserted in sequence at a restriction enzyme cloning site(s) of the gene transfer vector by restriction enzyme digestion and ligation (see Joseph Sambrook and David W. Russell, Molecular Cloning, the 3rd edition, 1.84-1.87, 2001). The pBlueBac4.5 vector derived from Autographa californica nuclear polyhedrosis virus (AcMNPV)(lnvitrogen, 1600 Faraday Avenue PO Box 6482 Carlsbad, Calif.) is used herein as a suitable gene transfer vector.

A linearized baculovirus genome and the baculovirus transfer vector (containing the first reporter gene, the IRES candidate sequence and the second reporter gene) are co-transfected into insect cells. In the insect cells, the desired expression cassette (containing the first reporter gene, the IRES candidate sequence and the second reporter gene) carried by the gene transfer vector may transfer into the baculovirus genome by homologous recombination, and thus a new recombinant baculovirus genome containing the desired expression cassette is obtained.

Baculoviruses are DNA-virus specific to invertebrate species such as insects and arachnids. Examples of baculovirus suitable for insect viral expression vectors include ACMNPV, PnMNPV (Perina nuda multinucleocapsid nuclear polyhedrosis virus), BmNPV (Bombyx mori nuclear polyhedrosis virus), LdMNPV (Lymantria dispar multinucleocapsid nuclear polyhedrosis virus) and OpMNPV (Orgyia pseudotsugata multicapsid nucleopolyhedrovirus). At least an essential gene involved in the virus replication in the baculovirus expression vector is deleted to prevent the baculovirus genome undergoing virus replication. Therefore, the baculovirus expression vector cannot produce any virus prior to the homologous recombination occurring between the gene transfer vector and the baculovirus genome to supply the missed essential gene for virus replication. The baculovirus genome is linearized and co-transfected with the transfer vector carrying the reporter genes and the IRES sequence candidate into an insect cell. The linearized baculovirus DNA and the transfer vector may undergo homologous DNA recombination, and thus the desired expression cassette (containing the first reporter gene, the IRES sequence candidate and the second reporter gene) are inserted into the virus genome following the promoter thereof to create a new recombinant virus. The new recombinant virus has the first reporter gene (the first cistron), the IRES sequence candidate, and the second reporter gene (the second cistron), operatively linked to the promoter in sequence, and initiates the transcription and translation mechanism of genes within the genome to produce virus particles.

There are several well-known methods of co-transfecting a gene transfer vector and a viral genome into insect cells. A suitable co-transfection method may be chosen depending on the needs of an operator or species and nature of cells, such as electroporation (Journal of General Virology, 70, 3501-3505, 1989) and liposome-mediated transfection (Methods in Cell Science, Springer Science+Business Media B.V., Formerly Kluwer Academic Publishers B.V.; Volume 22, Number 4, Dec. 2000, 257 - 263). For example, in liposome-mediated transfection, DNA may be incorporated in a liposome composed of phospholipids and then transferred into cells via fusion of the liposome with cell membrane. There are various commercial liposome transfection kits suitable for the present invention, such as Cellfectin™ Transfection Reagent (available from Invitrogen Corporation, 1600 Faraday Avenue, PO Box 6482, Carlsbad, Calif.). The described transfection methods and the determination of cell species are well known by any person skilled in the art.

After co-transfecting both the virus expression vector and the gene transfer vector into an insect cell, these two vectors perform homologous recombination to yield a recombinant virus expression vector containing the reporter genes and the IRES sequence candidate. The promoter on the recombinant genome drives the transcription mechanism to produce a single RNA product encoding both the first and second reporter genes and the IRES sequence candidate, which is between the two reporter genes. The first reporter gene encoded by the single RNA product is efficiently translated by cap-dependent translation mechanism. Therefore, the virus particles can be selected by the protein expression of the first reporter gene and a known method such as end-point dilution (Journal of General Virology, Vol 35, 393-396, 1977; O'Reilly D R, Miller L K & Luckow V A, 127, 1992, Baculovirus Expression Vectors, A Laboratory Manual, WH Freeman and Company, New York).

For the determination of the IRES activity, if the IRES sequence candidate can function as an IRES element, the second reporter gene is translated via the cap-independent translation driven by the IRES sequence candidate. Therefore, whether the IRES sequence candidate has IRES activity can be determined by the expression of the second reporter gene controlled by the IRES sequence candidate.

Examples of reporter genes for the selection of the recombinant virus and IRES activity indicator are, for example, genes of various fluorescent proteins such as EGFP or DsRed, tags such as His-tag, myc-tag, HA-tag or FLAG-tag, and enzymes such as luciferase or β-glactosidase. The detecting methods for various reporter genes are well known by any skilled person in the art.

Various methods may be performed for the detection of different reporter genes. For example, if a gene encoding a fluorescent protein is used as a reporter gene, the fluorescent protein expressed in an insect cell can be detected by fluorescent microscopy with excitation wavelength set at 488 nm for EGFP or at 558 nm for DsRed. Alternatively, if a sequence encoding a tag or a marker gene is used as a reporter gene, the SDS-PAGE electrophoresis and Western blot analysis can be used to detect the expressed tags or maker proteins with the aid of a suitable antibody. In some instances, substrates such as luciferin or o-Nitrophenyl-β-D-galacto-pyranoside (ONPG) that are suitable for detecting the expressed level of a reporter gene encoding luciferase or β-glactosidase are used. Detecting methods for other reporter genes are well known by any person skilled in the art (see Brasier A R, Tate J E, Habener J F., Biotechniques. 1989 Nov-Dec; 7 (10): 1116-22).

If the first and second reporter genes are simultaneously expressed in cells transfected with the bi-cistronic viral expression genome, then it can be concluded that the IRES sequence candidate possesses IRES activity. If only the first reporter gene is sufficiently expressed and the second reporter gene fails to express or the expression level is negligible, then it may conclude that the IRES sequence candidate does not possess IRES activity.

In some instances, said bi-cistronic viral expression genome can be further constructed so that a second IRES sequence candidate and a third reporter gene are operatively linked to the downstream of the second reporter gene in sequence. The expression of the third reporter gene can be detected according to the detecting methods described above. If all reporter genes including the first, the second and the third reporter genes, or both the first and the third genes are expressed, it may conclude that the second IRES sequence candidate has also possess IRES activity. On the contrary, if only the first and the second genes, bur not the third genes are expressed, or only the first gene is expressed, it may conclude that the second IRES sequence candidate does not possess any IRES activity (data not shown).

The term “operatively linked” means that DNA fragments of a promoter, an internal ribosome entry site (IRES) or other genes are sufficiently connected to direct and regulate the expression of genes.

The term “cistron” refers to a section of DNA or RNA that contains the genetic codes for a single polypeptide or a protein, and functions as a hereditary unit. The term “cistron” used herein is interchangeable with “gene”.

The term “I RES activity” refers to the ability of a polynucleotide sequence to drive cap-independent translational initiation by binding of the ribosome to internal ribosome entry site (IRES).

The term “homology or similarity” refers to the likeness or the percentage of identity between two sequences (e.g., polynucleotide or polypeptide sequences). Typically, the higher similarity of sequence means the more similarity of physical or chemical properties or biological activities.

According to the method of screening a polynucleotide with IRES activity described above, the present invention also provides a polynucleotide sequence with IRES activity (SEQ ID No.: 1), which comprises the PnV-5′-UTR, the nucleotides 1- 473 of perina nuda picorna-like virus genome (GenBank No. NC_{—}003113), as the following sequence:

(SEQ ID No.:1)
001 uuuuaaauau cggguacagg guuuuaaccc uguacccggu auucagaccu uagcuuuuga
061 gcuauuguaa gaagguagcc uagcuuuuaa gcaauggcgg uauuagaucu ugcuuuugag
121 cucuaucuag uacguguuua caauuaauuc gauuaguuaa gauuuuaauu aguuuuagua
181 accagugcuu caaucuucua uuguggcacu ggcuuggauc ucccuuacac augugauuac
241 augauagacu uauuaguagu agauacaucu aaauucuaca acgaccuagu aaguauuagu
301 uaugugaaau agaaugugga ggauuuuaaa uugugaauag gccuuuauau ucggaguagg
361 uaguauugcg uauacuauua aucccacaau acguggucuc cgucuuagua uuuuuaauuu
421 gcgccccaau ggaaauggcu cuucggacuu gaguacagag gggcaaccca uaa

The PnV-5′ UTR according to this invention may be a RNA or a single-stranded or double-stranded cDNA, and may have a single mutation, multiple mutations, deletions or truncations, provided that the IRES activity is maintained. The polynucleotides that are with about 70-95% of sequence identity, such as 70%, 75%, 80%, 85%, 90%, 95% or more, to that of the PnV-5′ UTR, are also encompassed within the scope of this invention.

In another aspect, the present invention provides a method of simultaneously expressing at least two polypeptides or proteins in an insect cell. The method comprises the following steps: selecting a suitable viral expression vector and a gene transfer vector; constructing the gene transfer vector to contain a first cistron, an IRES sequence selected by the method of this invention, and a second cistron in sequence; co-transfecting both the gene transfer vector and the viral DNA into a cell to undergo the DNA homologous recombination and to yield a new recombinant virus; infecting a host gene expression system (e.g., an insect cell or larva) with virus particles derived from the new recombinant virus; and detecting the expression of the first and second cistrons. The expressed proteins or peptides encoded by the first and second cistrons may be purified from the expression system for further analysis or applications. The recombinant virus expression vector is a bi-cistronic expression vector in which the first cistron is operatively linked to the promoter, and the IRES sequence is operatively linked to the 3′ end of the first cistron and the second cistron are operatively linked to the downstream of the IRES sequence. The IRES sequence comprises the PnV-5′ UTR or any variants thereof, provide that the IRES activity is maintained. The first and second cistrons may be any of the reporter genes, tag sequences and marker genes, or genes that encodes a desired protein or peptide.

Similarly, the viral expression vector may further comprise a second IRES sequence operatively linked to the second cistron and a third cistron operatively linked to the second IRES sequence to form a tri-cistronic expression vector (data not shown).

In yet another aspect, the present invention further provides a kit comprising at least one of the IRES sequences described above and an instruction manual for the use and the function of the IRES sequence.

### Embodiment 1

Construction of a Transfer Vector

The pIRES-EGFP plasmid (ClonTech, Palo Alto, Calif.) was amplified and purified according to the method described by Sambrook et al. (Joseph Sambrook and David W. Russell, Molecular Cloning, the 3rd edition, 8.18-8.24,2001) or any well-known methods in the art. The purified pIRES-EGFP plasmid was digested with restriction enzymes EcoR I and Sal I to obtain a DNA fragment (2.2 kb) containing both EMCV-IRES sequence and EGFP gene. The DNA fragment was then cloned into the EcoRI-Sal I cloning sites of an AcMNPV baculovirus gene transfer vector, pBlueBac4.5 (Invitrogen), to produce a plasmid named pBacIRE.

A polymerase chain reaction (PCR) was performed by using a synthetic primer containing a Nhe I site, another primer containing a EcoR I site and pDsRed1-N1 plasmid (BD Biosciences ClonTech, Palo Alto, Calif.) as a template to amplify the DsRed gene fragment. The sequence of the primer containing a Nhe I site (underlined) was 5′ATCGGCTAGCGGCCACCAT GGTGCGCTCT (SEQ ID No.: 2) and the sequence of the primer containing a EcoR I site (underlined) was 3′GTAGGAATTCGCTACAGGAACAGGTGGTGG (SEQ ID No.: 3). The resulted PCR product, the DsRed gene fragment containing a Nhe I site at 5′ end and an EcoR I site at 3′ end, was then cloned into the pBacIRE plasmid to obtain a plasmid named pBacDIRE. After replacing the EMCV-IRES of the pBacDIRE plasmid with the IRES sequence candidate according to this invention or other IRES sequence, the pBacDIRE plasmid was used for an IRES activity analysis, wherein the DsRed gene was chosen as the first reporter gene and the EGFP gene was chosen as the second reporter gene.

The CrPV IGR-IRES (nucleotides 1-247 of GenBank No. NC003924) was obtained by chemical synthesis or any other well-known DNA preparation method in the art. It has been reported that the CrPV IGR-IRES represented IRES activity in a cell-free insect expression system (e.g. in-vitro transcription/translation system), but the CrPV IGR-IRES did not represent IRES activity in an insect, an insect cell or tissue (in vivo) (Wilson et al., Mol. Cell Biol. 20: 4990-4999, 2000). Therefore, the CrPV IGR-IRES was used herein as a negative control for IRES activity assay.

The CrPV IGR-IRES fragment containing both an EcoR I site at 5′ end and a BamH I site at 3′ end was obtained from MDBio Inc (Taiwan). The CrPV IGR-IRES fragment was subcloned into pBacDIRE plasmid processed with both EcoR I and BamH I restriction enzymes to create a new plasmid named “pBacDCrirE”. The resulted pBacDCrirE plasmid had a DsRed gene and an EGFP gene separated by the CrPV IGR-IRES fragment.

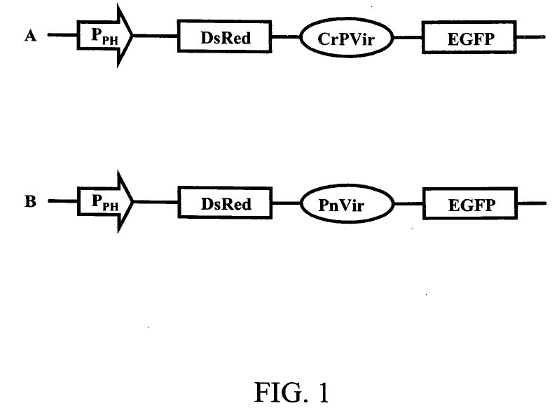

According to the method described in Wu et al. Virology, 294: 312-323, 2002, the PnV genomic RNA was extracted from the purified perina nuda picorna-like virus by using TRIzol® Reagents (Invitrogen Corp., Carlsbad, Calif.). An RT-PCR was performed to amplify the cDNA of PnV 5′ UTR by using a forward primer 5′-GCGGA TCCTT TTAAA TATCG GGTAC AGGGT TTTAA CC-3′ (the 1-29 nts of PnV genome) (SEQ ID NO.: 4), a reverse primer 5′-GCGGA TCCTT ATGGG TTGCC CCTCT GTACTC-3′ (complementary to the 451-473 nts of PnV genome) (SEQ ID No.: 5), PnV genomic RNA as a template and RT-PCR kit such as Superscrip™ One-step RT-PCR Reagent for long template available from Invitrogen (Invitrogen). The RT-PCR products were directly cloned into pGEM®-T Easy Vector (Promega, 2800 Woods Hollow Road, Madison, Wis. 53711 USA). The sequence of the PnV 5′ UTR cDNA inserted in the pGEM®-T Easy Vector was confirmed by DNA sequencing according to standard technique. Then, the PnV 5′ UTR cDNA (473 units) was released by BamH I digestion and inserted at the BamH I site of the bi-cistronic plasmid pBacDirE between the two reporter genes (FIG. 1). The orientation of the PnV 5′ UTR cDNA was determined by sequencing with the primer 5′-CTACG TGGAC TCCAA GCTGG-3′ (derived from the downstream sequence of the DsRed gene) (SEQ ID No: 6). The created plasmid containing the PnV 5′ UTR in the sense orientation was selected and named “pBacDPnirE”. The pBacDPnirE plasmid has DsRed and EGFP genes that were separated by the PnV 5′ UTR.

Preparation and Titration of Recombinant Viruses

S. frugiperda IPBL-Sf21 insect cell line (hereinafter Sf21 cells) was cultured in TNM-FH medium containing 8% of heat-inactivated FBS until a confluent cell monolayer (about 2×10⁵ cells/well) was obtained. The pBacDPnirE or pBacDCrirE plasmid (0.8 μg) was transfected into the confluent Sf21 insect cells together with the linearized Bac-N-Blue baculovirus DNA (0.25 μg) by using 1 μl of Cellfectin™ Transfection Reagent (Invitrogen Corp., Carlsbad, Calif.). The transfected cells were cultured in TNM-FH medium free of FBS for 12 hours, and then were cultured in TNM-FH medium containing 8% heat inactivated FBS.

The Bac-N-Blue baculovirus DNA and the pBacDPnirE or pBacD-CrirE gene transfer vector might have undergone the DNA homologous recombination to generate a new recombinant baculovirus expression vector containing the DsRed gene, the IRES sequence (such as PnV 5′ UTR) and the EGFP gene.

The resulted recombinant baculovirus has the ability of expressing the DsRed fluorescent protein, and therefore, on Day 6 after the co-transfection, the cells containing the recombinant virus expression could be recognized and selected according to the presence of the RsRed fluorescent protein under a fluorescence microscopy (Nikon, Japan). The transfected cells produced a lot of the recombinant virus particles. The desired recombinant viruses can be selected by end-point dilution method (Journal of General Virology, Vol 35, 393-396, 1977; O'Reilly D R, Miller L K & Luckow V A, 127, 1992, Baculovirus Expression Vectors, A Laboratory Manual, WH Freeman and Company, New York), and named as vAcD-Crir-E and vAcD-Pnir-E.

FIG. 1 was the schematic plasmid constructions of these two recombinant virus expression vectors vAcD-Crir-E (A construction) and vAcD-Pnir-E (B construction), illustrating the polyhedron promoter (P_{PH}) of the virus genome, which was sequentially followed by the DsRed gene, the CrPV IGR-IRES (for A construction) or the PnV-5′UTR (for B construction), and the EGFP gene. The recombinant vAcD-Crir-E virus was used as a negative control for the following IRES activity assay.

The titer of these two recombinant viruses, vAcD-Crir-E and vAcD-Pnir-E, was determined by end-point dilution and DsRed fluorescence was detected according to the steps described by O'Reilly et al. (O'Reilly D R, Miller L K & Luckow V A, 127, 1992, Baculovirus Expression Vectors, A Laboratory Manual, WH Freeman and Company, New York), and the virus titer thereof was also calculated according to the 50% tissue culture infectious dose 9TCID₅₀).

PnV 5′ UTR Displays IRES Activity in Sf21 Cells

The Sf21 (or Sf9) insect cells were cultured in TNM-FH medium containing 8% heat-inactivated FBS until the cultures approximately formed a confluent monolayer. The monolayer cells were then infected with the purified vAcD-Crir-E or vAcD-Pnir-E virus. The promoter (P_{PH}) of these two virus genome drove a transcription mechanism to produce a single RNA product encoding the DsRed gene, the CrPV IGR-IRES or the PnV-5′UTR, and the EGFP gene therein in the infected Sf21 cells.

In the first embodiment, a Northern blot assay was performed to determine the size of the single RNA product by using a RNA probe specific to the EGFP gene within the vAcD-Pnir-E virus genome. For the Northern blot assay, the Sf9 insect cells were first infected with or without the vAcD-Pnir-E recombinant virus. On Day 7 after infection, the total RNAs were then extracted from the infected and uninfected Sf9 insect cells according the standard ENA extraction method well known in the art.

For preparing the RNA probe specific to EGFP gene, a EGFP DNA fragment (366 bps) was obtained from the pBacDpnirE plasmid by PCR using a forward primer EGFP-F (5′-ACGAC TTCTT CMGT CCGCC-3′) (SEQ ID No.: 7) and a reverse primer EGFP-R (5′-TGCTC AGGTA GTGGT TGTCG-3′) (SEQ ID No.: 8). The EGFP DNA fragment (366 bps) was then cloned into a pGEM-T Easy Vector™ containing T7/SP6 promoters (Promega Corporation, 2800 Woods Hollow Road Madison, Wis. 53711 USA). An in-vitro transcription was performed using the PGEM-T Easy VectorTM containing EGFP gene and DIG-RNA Labeling Kit (Roche, Grenzacherstrasse 124, CH-4070 Basel, Switzerland), to obtain a DIG-labeled RNA probe specific to EGFP gene. The RNA transcripts were extracted from the vAcD-Pnir-E infected (3 days post infection) and the uninfected Sf9 cells and then analyzed by 1% argarose-formaldehyde gel electrophoresis. The RNA in the gel was then transferred to a Hybond-NTM nylon membrane (Amersharn Biosciences Corp, 800 Centennial Avenue, P.O. Box 1327, Piscataway, N.J. 08855-1327, USA). The membrane was probed with the DIG-labeled RNA probe according to the northern blot protocol described by Sambrook et al. (Joseph Sambrook and David W. Russell, Molecular Cloning, the 3rd edition, 7.31-7.44,2001). Standard chemiluminescent detection was performed according to the manufacturer's instructions (Roche), and the membrane was exposed to X-ray film (Kodak XAR-5) to determine the molecular size of the RNA transcripts derived from the vAcD-Pnir-E virus genome.

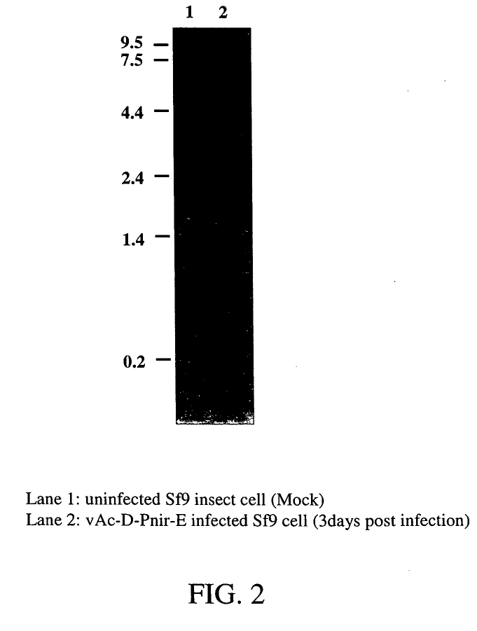

FIG. 2 shows the Northern blot results of the uninfected and vAcD-Pnir-E infected Sf9 cells. In FIG. 2, no virus genomic RNA transcripts were detected for the uninfected Sf9 cells (lane 1), while a single RNA transcript with a size of 2.4 kb was detected by the DIG-labeled probe in the total RNAs of the vAcD-Pnir-E infected cells (3 days post infection). It indicated that the vAcD-Pnir-E virus genome was transcribed to a single RNA product containing all of the DsRed/EGFP genes and the PnV-5′UTR sequence.

The DsRed gene encoded by the vAcD-Crir-E or vAcD-Pnir-E recombinant virus genomic RNA was efficiently translated by cap-dependent translation mechanism. However, the expression of the EGFP gene encoded by the virus RNA transcripts depends on the cap-independent translation mechanism. Therefore, the presence of the EGFP protein indicated whether the CrPV IGR-IRES or the PnV-5′ UTR represents IRES activity in insect cells.

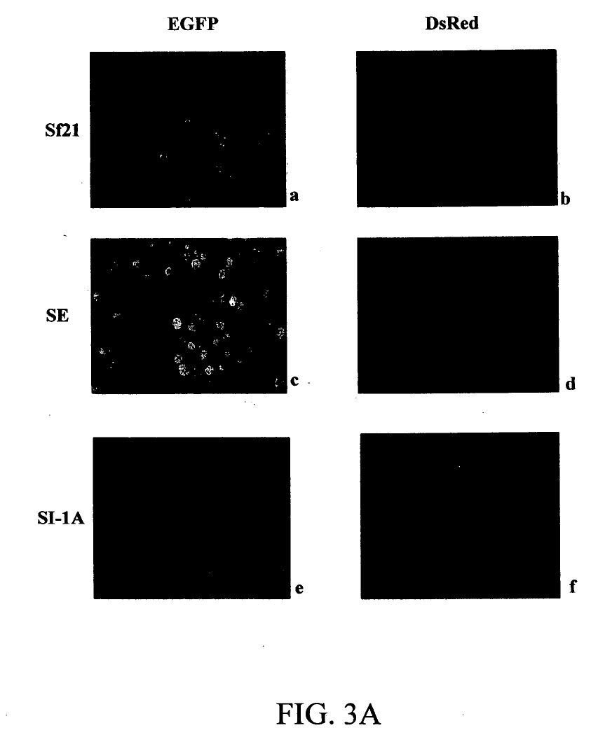

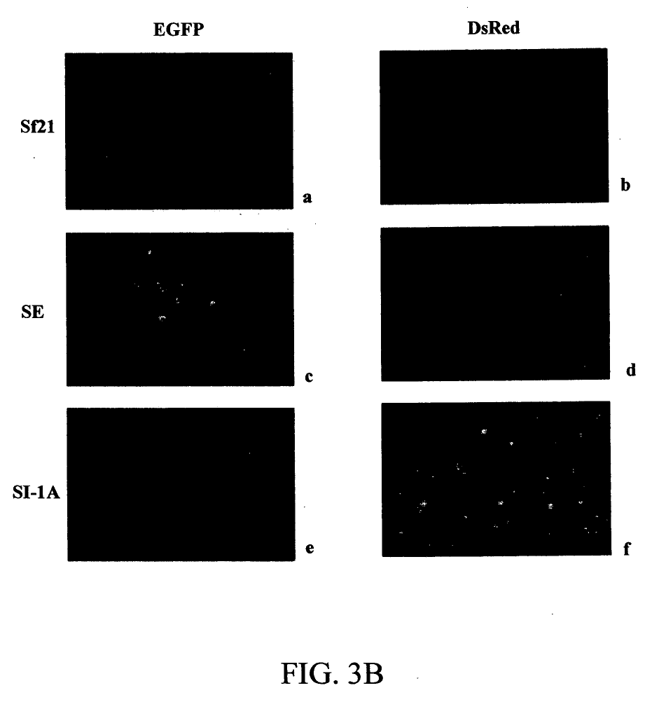

FIG. 3A illustrates the protein expression level of EGFP (a) and DsRed (b) in Sf21 cells infected with vAcD-Pnir-E virus. FIG. 3B illustrates the expression level of EGFP (a) and DsRed (b) in Sf21 cells infected with vAcD-Crir-E virus.

The Sf21 insect cells infected with vAcD-Crir-E or vAcD-Pnir-E virus were cultured in suitable medium for several days, and then were observed under fluorescence microscopy (Nickon, Japan) by exciting with green light (Rhodamin filter) for DsRed and exciting with blue light (FITC filter) for EGFP. FIGS. 3A and 3B were fluorescence photography respectively illustrating the DsRed/EGFP protein expression level in the vAcD-Pnir-E or vAcD-Crir-E infected cells. In FIGS. 3A and 3B, both of the vAcD-Crir-E or vAcD-Pnir-E infected Sf21 cells (b) emitted red fluorescence, which indicated that the DsRed gene was efficiently expressed by cap-dependent translation initiation mechanism for vAcD-Crir-E or vAcD-Pnir-E recombinant viruses.

In FIG. 3A, the PnV-5′UTR within the vAcD-Pnir-E virus genome sufficiently drove the Cap-independent translation initiation of the EGFP gene in Sf21 insect cells, and thereby the vAcD-Pnir-E infected Sf21 cells emitted green fluorescence (a). Referring to FIG. 3B, the CrPV IGR-IRES sequence of the vAcD-Crir-E virus genome failed to drive the Cap-independent translation initiation of the EGFP gene in Sf21 insect cells, and thus the vAcD-Pnir-E infected Sf21 cells do not emit any green fluorescence (a). Therefore, it suggested that the PnV-5′ UTR might possess IRES activity, due to the reason that both EGFP and DsRed proteins were present in vAcD-Pnir-E infected cells.

Western Blot

For Western blot assay, Sf21 insect cells were infected with the purified vAcD-Crir-E or vAcD-Pnir-E viruses and then cultured in a cell-culture medium for several days (e.g. 4 days). The infected cells were then harvested and lysed with lysis buffer to obtaine the cell lysates. The proteins of the cells lysates were separateded by SDS-PAGE. The proteins in the gel were then transferred on a polyvinyldiene difluoride membrane (PVDF, Millipore). The membrane was then blocked with a Tris-buffer (100 mM Tris, pH7.4; 100 mM NaCI and 0.1% Tween 20) containing 5% bovine serum albumin (BSA, Sigma) at room temperature for 1 hour.

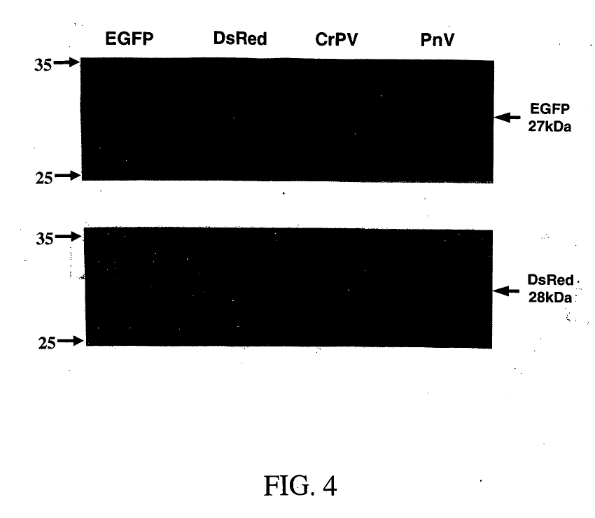

After blocking, the membrane was incubated with an anti-EGFP antibody (1:2000, BD Biosciences ClonTech, Palo Alto, Calif.) at 4° C. overnight. The membrane was then washed with Tris-buffer at room temperature three times, each time for 5 minutes, to remove the unbonded anti-EGFP antibody. The PVDF membrane was then incubated with horseradish peroxidase (HRP) conjugated secondary antibody (1:2500, Jackson) at room temperature for 1 hour. The PVDF membrane was washed with the same Tris-buffer at room temperature three times, each time for 5 minutes to remove any unbonded secondary antibody. The EGFP protein on the PVDF membrane was detected by using Enhanced Chemiluminescence Kit (Piece) (shown in the upper panel of FIG. 4).

After stripping the membrane to remove the anti-EGFP antibody, the PVDF membrane was incubated with an anti-DsRed antibody (BD Biosciences ClonTech, Palo Alto, Calif.) Divulged with PBS buffer in a ratio of 1: 2000 at 4° C. overnight. The PVDF membrane was washed with Tris-buffer at room temperature three times, and each time for 5 minutes, to remove any unbonded antibodies. The PVDF membrane was then incubated with the horseradish peroxidase (HRP) conjugated secondary antibody (1:2500, Jackson) at room temperature for 1 hour. The PVDF membrane was washed with the same Tris-buffer at room temperature three times, each time for 5 minutes to remove any unbonded secondary antibody. The DsRed protein on the PVDF membrane was detected by using Enhanced Chemiluminescence Kit (Piece) (shown in the lower panel of FIG. 4).

The order of detecting these two proteins may be altered. The time for reaction or washing, compositions of buffers or other conditions used herein are illustrated by example, and can be modified based upon any special concerns, needs or desires.

FIG. 4 is a Western blot illustrating the protein expression of DsRed and EGFP gene in the Sf21 cells respectively infected with vAcD-Crir-E or vAcD-Pnir-E virus. In FIG. 4, lanes 1 and 2 referred to EGFP and DsRed, respectively, were positive controls for EGFP (27 kDa) and DsRed (28kDa) proteins. Lane 3 (CrPV) was a negative control for showing that the CrPV IGR-IRES sequence within the vAcD-Crir-E virus genome failed to drive Cap-independent translation initiation (i.e. cap-independent translation initiation), and therefore lane 3 (CrPV) proved that only the DsRed fluorescent protein was expressed in the vAcD-Crir-E infected cells, but no EGFP protein was expressed. Lane 5 (PnV) showed that the vAcD-Pnir-E infected cells expressed both DsRed and EGFP fluorescent proteins, and therefore, it was suggested that the PnV 5′ UTR according to the present invention was capable of driving the Cap-independent translation initiation mechanism of the EGFP gene. Briefly, the PnV 5′UTR according to this invention had IRES activity.

The result of this Western blot assay corresponds to the protein expression level observed under fluorescent microscopy and indicated that the 5′ UTR of perina nuda picorna-like virus genome (PnV) possesses IRES activity.

### Embodiment 2

Construction of Recombinant Viruses vAcD-Rhir-E and vAcD-Pn539ir-E

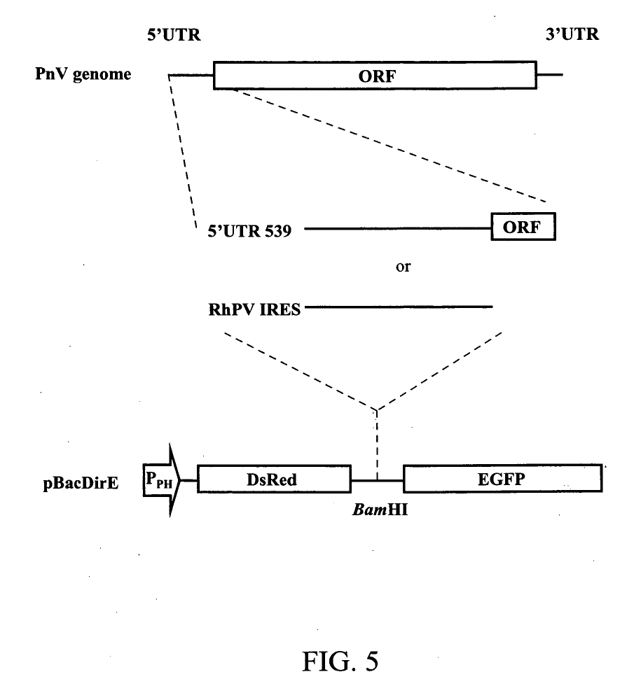

It has been reported that the downstream sequence of the HCV-IRES sequence is capable of regulating the cap-independent translation activity of the HCV-IRES (Wang, et al., J. Viology 74: 11347-11358, 2000). In order to determine whether the downstream sequence of the PnV 5′ UTR (also referred to as PnV-IRES sequence) enhances the IRES activity, a plasmid construction containing both the PnV 5′UTR and the downstream sequence thereof was constructed for this study (shown in FIG. 5). For this construction, a DNA fragment (539 nts) containing both PnV 5′UTR (473 nts) and the first 22 codons (66 nts) of the PnV-ORF region was amplified by RT-PCR from the PnV genomic RNA using a forward primer (PnV-F539) 5′-GCGGA TCCTT TTAAA TATCG GGTAC AGGGT TTTAA CC-3′ (SEQ ID No.: 4) and a reverse primer PnV-R539 5′-GGTGG ATCCG TGCGA AAGTT CGTCA G-3′ (SEQ ID No.: 9), each containing a BamH I site, while the IRES sequence of Rhopalosiphum padi virus (RhPV-IRES) was used as a control in the second embodiment (see Chen et al., Biochemical and Biophysical Research Communication 335:616-623, 2005). The RhPV-IRES and the DNA fragment containing both the PnV-IRES and the first 22 codons of PnV-ORF were respectively inserted into the BamH I site of baculovirus transfer vector pBacDirE described in the embodiment 1 to create two new transfer vectors named pBacD-RhirE and pBacD-Pn539irE. These two gene transfer vectors pBacD-RhirE and pBacD-Pn539irE were then co-transfected into an insect cell together with the linearized Bac-N-Blue™ baculovirus expression vector for DNA homologous recombination, to generate two recombinant viruses named vAcD-Rhir-E and vAcD-Pn539ir-E, respectively.

The first 22 codons (66 nts) of the PnV-ORF region of perina nuda picorna-like virus genome (GenBank No. NC_{—}003113) according to this present invention, i.e. the nucleotides 474-539 of perina nuda picorna-like virus genome, are provided as the following sequence:
474 augauga uuaacccaca acaauuaugu aagaaaacac uuucugacga acuuugcgau cgcacggau (SEQ ID No.: 10)
   IRES Activity Analysis for PnV 5′ UTR (539nt)

Sf21 cells (in 24-well plate) were cultured in TNM-FH medium containing 8% heat-inactivated FBS until the cultures were approximately confluent (2×10⁵/well). The cells were then infected with the recombinant virus vAcD-Crir-E, vAcD-Pnir-E, vAcD-Rhir-E or vAcD-Pn539ir-E. On Day 4 post infection with each virus, the cells were observed under microscopy (Nickon, Japan) by exciting with green light (Rhodamin filter) for DsRed and blue light (FITC filter) for EGFP to determine the expression of the DsRed and EGFP genes. In the remaining figures, the cells infected with vAcD-Crir-E virus are referred to as CrPV; the cells infected with vAcD-Pnir-E virus are referred to as PnV; the cells infected with vAcD-Pn539ir-E virus are referred to as PnV5′539; and the cells infected with vAcD-Rhir-E virus are referred to as RhPV.

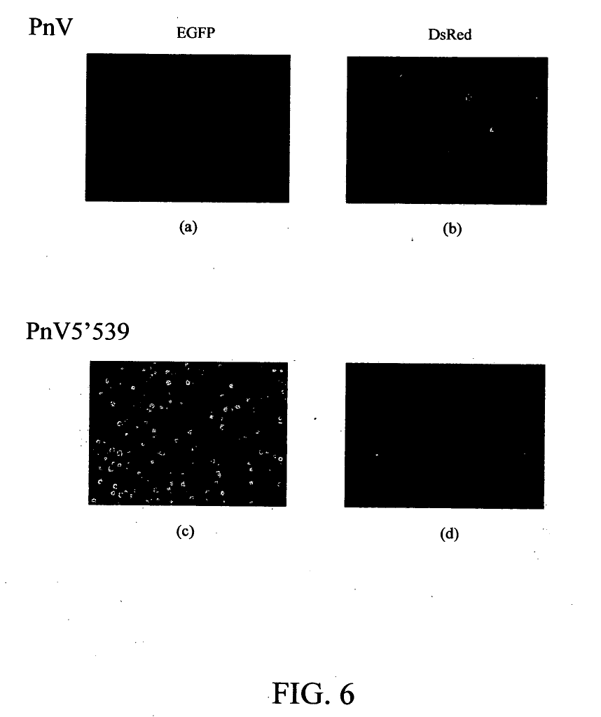

Referring to FIGS. 6a-6b, the PnV 5′ UTR (473 nts) represents the IRES activity and mediated the cap-independent translation initiation to express both EGFP protein (FIG. 6a) and DsRed protein (FIG. 6b) in Sf21 insect cell. As shown in FIG. 6, the PnV genomic fragment (539 nts) containing PnV 5′ UTR (473nt) and the first 22 codons of PnV-ORF also showed the IRES activity according to the presence of both EGFP (FIG. 6c) and DsRed proteins (FIG. 6d) in Sf21 cells. Moreover, after comparing the green fluorescence intensity of FIGS. 6a and 6c, the PnV genomic fragment (539 nts) containing both PnV 5′ UTR (473nt) and the first 22 codons of PnV-ORF showed IRES activity stronger than the PnV 5′ UTR.

Comparison and Quantification of IRES Activity

For the comparison of IRES activity between the PnV 5′ UTR (473 nts) and PnV-genomic fragment 539 (539 nts), the fluorescence intensity of the cells respectively infected with said four recombinant viruses were quantified. First, the infected cells were treated with 300 pl of lysis buffer containing 100 mM potassium phosphate (pH 7.8), 1 mM EDTA, 1% Triton X-100 and 7 mM β-mercaptoethanol for 10 minutes. The lysed cells were centrifuged at 12,800 rpm for 30 minutes to obtain the supernatant.

In order to determine the fluorescence intensity, 100 μl of each supernatant was detected by using Cary Eclipse Fluorescence Spectrophotometer (Varian Instruments, Walnut Creek, Calif.). The EGFP protein in each supernatant was excited by 488 nm light, while the DsRed protein was excited by 558 nm light. The emitted 507 nm light and the 583 nm light were respectively quantified for green and red relative fluorescent unit (RFU). The red and green fluorescence intensities were calculated from the green and red RFU value to indicate the EGFP and DsRed protein expression level in each kind of infected cell.

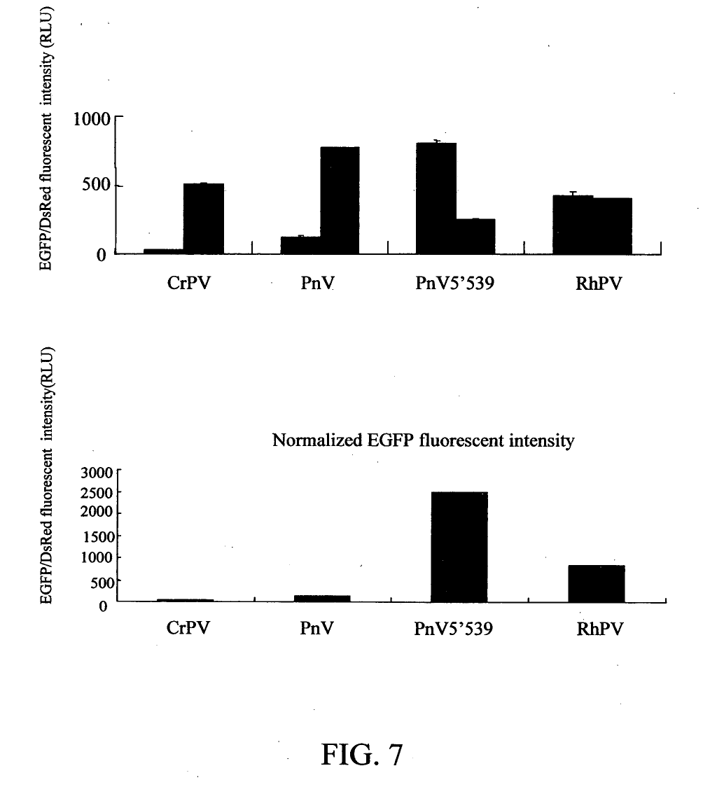

The quantitative results of the fluorescence intensity assay are shown in FIG. 7. In FIG. 7, the y-axis represents green/red fluorescence intensity; x-axis represents IRES sequence candidate s used in this assay; the light gray bar represents intensity of green fluorescence; and the dark gray bar represents intensity of red fluorescence. FIG. 7 shows the ability of directing cap-independent translation initiation (IRES activity) for four candidate/control IRES elements, CrPV IGR-IRES (CrPV), PnV-5′ UTR (PnV), polynucleotide containing both PnV 5′UTR and the first 66 nucleotides of PnV-ORF (PnV-5′539) and RhPV-IRES (RhPV). In the upper panel of FIG. 7, the CrPV IGR-IRES element, which failed to mediate the cap-independent translation initiation in insect cells, was a negative control; on the contrary, the RhPV-IRES (RhPV) was a positive control for IRES activity. The PnV 5′ UTR (473 nts) according to the present invention represented IRES activity to mediate EGFP protein expression by cap-independent translation mechanism (lane 2 referred as PnV). However, the polynucleotide containing PnV 5′ UTR and the first 66 nucleotides of the PnV-ORF showed significantly stronger IRES activity than PnV 5′ UTR alone. This quantification of fluorescence intensity shown in FIG. 7 was performed again by normalization. For the normalization, the amount of DsRed fluorescence protein presence in each of the lysates was detected by spectrofluometer. A given volume of each of the diluted lystaes were detected and calculated for normalized fluorescence intensity in the same manner as described above. The result established that the IRES sequence candidate having both PnV 5′ UTR and the first 22 codons of PnV-ORF according to the present invention had significant increased IRES activity stronger than the other three IRES sequences did (lane 3 referred to as PnV5′539 in the lower panel of FIG. 7).

Western Blot

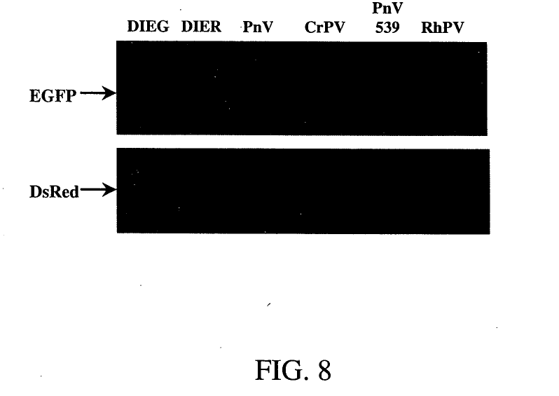

The protein expression level of DsRed and EGFP were determined by Western blot technique as described in the embodiment 1 and shown in FIG. 8. Referring to FIG. 8, lanes labeled DIEG and DIER were controls for illustrating the molecule sizes of green and red fluorescence proteins, respectively. Lane RhPV was positive control for IRES activity. According to FIG. 8, the PnV 5′ UTR in the recombinant virus vAcD-Pnir-E directs the cap-independent translation initiation of the downstream EGFP gene, and thus the green fluorescence was observed under the microscope with FITC filter in FIG. 6. In particular, corresponding to the fluorescence intensity quantification shown in FIG. 7, the recombinant virus vAcD-Pn539ir-E (PnV 5′539) containing both the PnV 5′-UTR and the first 22 codons of PnV-ORF represents a significantly higher protein expression level than those of the other three recombinant virus (referred as CrPV, PnV and RhPV, respectively).

The result in the embodiment 2 not only showed that the PnV 5′ UTR represents IRES activity, but also established that the first 22 codons of the PnV-ORF (i.e. the first 66 nucleotides of the PnV-ORF) are critical for IRES activity. In embodiment 2, the polynucleotide sequence comprising both PnV 5′ UTR and the first 22 codons of PnV-ORF had strong IRES activity.

### Other Embodiments

IRES analysis was also performed by using NTU-SE cells derived from Spodoptera exigua and NTU-SL1A cells derived from Spodoptera litura. In FIG. 3A, the recombinant virus vAcD-Pnir-E expressed the first cistron, DsRed, via cap-dependent translation mechanism, and also efficiently expressed the second cistron, EGFP, by cap-independent translation mechanism. Therefore, the vAcD-Pnir-E transfected NTU-Se and NTU-SL1A cells emitted red fluorescence (d and f) as well as green fluorescence (c and e) simultaneously. Further referring to FIG. 3B, the CrPV IGR-IRES element of vAcD-Crir-E virus genome failed to lead the cap-independent translation initiation of the second cistron, EGFP gene, and thus the vAcD-Crir-E infected NTU-SE and NTU-SL1A cells emitted the red fluorescence (d and f) only, but not the green fluorescence (c and e). The gray or faint green spots in FIG. 3B (c) and (e) were noise from the red light under FITC filter.

This result was consistent with the foregoing IRES activity analysis in Sf21 cells, and indicated that the 5′ UTR of genome of perina nuda picorna-like virus (PnV) directed mRNA containing it therein to undergo cap-independent translation.

The PnV-5′ UTR can be replaced with any IRES sequence candidate. The same method described above or any other method well known in the art can be performed to determine whether an IRES sequence candidate directs cap-independent translation initiation in an insect expression system according to the presence or absence of the DsRed/EGFP proteins.

In the same manner, when the DsRed/EGFP genes are replaced with other marker genes or sequences encoding a protein or a peptide, the method of simultaneously expressing at least two proteins or peptides in an insect expression system is achieved by using only one viral expression vector with the IRES polypnucleotide according the present invention.

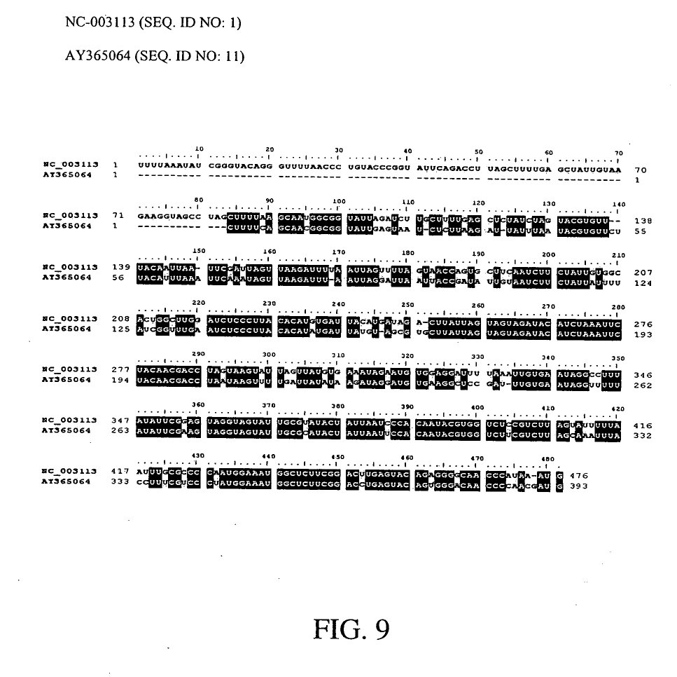

Similar sequence might imply similar structure, and most of the time, similar structure means similar functionality. Therefore, an IRES sequence candidate can be selected according to the similarity in sequence alignment with a known IRES sequence. For example, genome sequences of other viruses can be screened by comparison with that of the 5′ UTR of perina nuda picorna-like virus for further IRES activity analysis. The PnV 5′ UTR with IRES activity according to the present invention (GenBank No. NC_{—}003113) (SEQ ID No. 1) represents a high sequence homology with the 5′ UTR of Ectropis obliqua picorna-like virus (GenBank No. AY365064) (SEQ. ID NO: 11) as shown in FIG. 9. It is reasonable to presume that the 5′ UTR of Ectropis obliqua picorna-like virus may have IRES activity which can be determined according to the methods described in the above-mentioned embodiments.

While the foregoing is directed to embodiments of the present invention, other and further embodiments of the invention may be devised without departing from the basic scope thereof, and the scope thereof is determined by the claims that follow.

What is claimed is:

1. An isolated polynucleotide sequence with IRES activity for directing a Cap-independent translation in an insect expression system, comprising 5′-UTR of genome of perina nuda picorna-like virus (PnV 5′UTR) or a variant thereof.

2. The isolated polynucleotide sequence of claim 1, wherein the isolated polynucleotide sequence comprises the nucleotides 1-473 of genome of perina nuda picorna-like virus (SEQ ID No.: 1).

3. The isolated polynucleotide sequence of claim 2, further comprising the nucleotides 474-539 of genome of perina nuda picorna-like virus (SEQ ID No.: 10).

4. The isolated polynucleotide sequence of claim 1, wherein the polynucleotide is a RNA, a single stranded cDNA or a double stranded cDNA.

5. The isolated polynucleotide sequence of claim 1, wherein the variant is the polynucleotide with a single point mutation, multipoint mutations, truncation or deletion or a combination thereof.

6. The isolated polynucleotide sequence of claim 1, wherein the variant is at least 70%-90% identical to the sequence of PnV 5′-UTR.

7. The isolated polynucleotide sequence of claim 1, wherein the insect expression system is selected from a group consisting of an insect, an insect cell, an insect tissue or an cell-free insect expression system.

8. A kit comprising the isolated polynucleotide sequences of any of claims 1 to 3 and an instruction manual for the use and the function of the polynucleotides.

9. A method of expressing at least two polypeptides in an insect expression system, comprising:
selecting a viral expression vector;
constructing the viral expression vector to comprise a promoter operably linked to a polynucleotide sequence comprising at least two cistrons, at least a polynucleotide sequence with IRES activity according to any of claim 1 to 3;
introducing the viral expression vector into an insect expression system; and
detecting the expression of the at least two cistrons in the insect expression system.

10. The method of claim 9, wherein the insect expression system is selected from a group consisting of an insect, an insect cell, an insect tissue and an cell-free insect expression system.

11. The method of claim 9, wherein the viral expression vector is a beculovirus expression vector.

12. The method of claim 11, wherein the beculovirus expression vector is AcMNPV, PnMNPV, BmNPV, LdMNPV or OpMNPV.


SEQUENCE LISTING (11 sequences)

SEQ ID NO: 1 - RNA, 473 nt, Perina nuda picorna-like virus
uuuuaaauaucggguacaggguuuuaacccuguacccgguauucagaccuuagcuuuugagcuauuguaagaagguagccuagcuuuuaagcaauggcgguauuagaucuugcuuuugagcucuaucuaguacguguuuacaauuaauucgauuaguuaagauuuuaauuaguuuuaguaaccagugcuucaaucuucuauuguggcacuggcuuggaucucccuuacacaugugauuacaugauagacuuauuaguaguagauacaucuaaauucuacaacgaccuaguaaguauuaguuaugugaaauagaauguggaggauuuuaaauugugaauaggccuuuauauucggaguagguaguauugcguauacuauuaaucccacaauacguggucuccgucuuaguauuuuuaauuugcgccccaauggaaauggcucuucggacuugaguacagaggggcaacccauaa

SEQ ID NO: 2 - DNA, 29 bp, Artificial sequence
  Synthetic Primer
atcggctagcggccaccatggtgcgctct

SEQ ID NO: 3 - DNA, 30 bp, Artificial sequence
  Synthetic Primer
gtaggaattcgctacaggaacaggtggtgg

SEQ ID NO: 4 - DNA, 37 bp, Artificial sequence
  Synthetic Primer
gcggatccttttaaatatcgggtacagggttttaacc

SEQ ID NO: 5 - DNA, 31 bp, Artificial sequence
  Synthetic Primer
gcggatccttatgggttgcccctctgtactc

SEQ ID NO: 6 - DNA, 20 bp, Artificial sequence
  Synthetic Primer
ctacgtggactccaagctgg

SEQ ID NO: 7 - DNA, 20 bp, Artificial sequence
  Synthetic Primer
acgacttcttcaagtccgcc

SEQ ID NO: 8 - DNA, 20 bp, Artificial sequence
  Synthetic Primer
tgctcaggtagtggttgtcg

SEQ ID NO: 9 - DNA, 26 bp, Artificial sequence
  Synthetic Primer
ggtggatccgtgcgaaagttcgtcag

SEQ ID NO: 10 - RNA, 66 nt, Perina nuda picorna-like virus
augaugauuaacccacaacaauuauguaagaaaacacuuucugacgaacuuugcgaucgcacggau

SEQ ID NO: 11 - RNA, 393 nt, Ectropis obliqua
cuuuucagcaacggcgguauugaguaaucucuuaagauuauuuaauacguguucuuacauuuaaauucaaauaguuaagauuuaauuaggauuaauuaccgauauuguaaucuucuauuauuuuaucgguuugaaucucccuuacacauaugauuauguagcgugcuuauuaguaguagauacaucuaaauucuacaacgaccuaauaaguuuugauuauauaagauaggaugugaaggcuccgauuugugaauagguuuuuauauucgaaguagguaguauugcgcauacuauuaauuccacaauacguggucuucgucuuagcaaauuuaccuuucgucccuauggaaauggcucuucggaccugaguacagugggacaaccccaacgaug